(19) United States
(12) Patent Application Publication
(10) Pub. No.: US 2002/0035385 A1
(43) Pub. Date: Mar. 21, 2002

(54) Connector head for, and process of joining same to the case of an active implantable medical device such as a pacemaker, a defibrillator, a cardiovertor and/or multisite device

(76) Inventor: Marc Magid Deziz, Pontarlier (FR)

Correspondence Address: Robert M. Isackson, ESQ, ORRICK, HERRINGTON & SUTCLIFFE LLP, 666 Fifth Avenue, New York, NY, 10103-0001, US

(21) Appl. No.: 09/903,401

(22) Filed: Jul. 11, 2001

(30) Foreign Application Priority Data
Jul. 11, 2000 (FR) 00 09053

(51) Int. Cl. A61N 1/375

(52) U.S. Cl. 607/37

(57) ABSTRACT

A connector head (10) and a process of joining a connector head to the case (12) of an active implantable medical device, such as a pacemaker, defibrillator, cardiovertor and/or a multisite device. The process includes the steps of a) mechanically and electrically assembling the connector head onto the case; b) injecting of a quantity of a photo-hardening adhesive, in particular a photo-reticulable silicone resin, into an interface formed between the assembled case and the connector head; c) irradiating the quantity of adhesive by a light having a wavelength that is adapted to harden the adhesive; d) optionally affixing a mask on a portion of the case; e) immersing the case provided with its connector head into a photo-hardening resin, so as to form on the case and the connector head a layer of photo-hardening resin coating; and f) irradiating this layer of resin by a light having a wavelength adapted to harden the resin. The connector head (10) is made out of a transparent or translucent material, and it comprises a peripheral lip (18) having a whose surface (20, 22) that mates to the case so as to define a closed volume (24) in an interface between the case and the connector head. The head has an injection opening at one extremity of the closed volume to inject adhesive (14) into the interface and at least one vent opening (16) communicating with the closed volume (24) at another extremity to allow the adhesive to fill the closed volume prior to curing.

### FIELD OF THE INVENTION

The present invention relates to the manufacture of active implantable medical devices.

Although the invention will be mainly described in the context of a pacemaker, it should be understood that this is only one exemplary embodiment of an invention that is applicable in a much more general way to a very large variety of “active implantable medical devices” as such devices are defined by the Jun. 20, 1990 Directive 90/385/CE of the Council of the European Communities. These devices thus include, in addition to pacemakers, and among other devices, defibrillators, cardiovertors, neurological apparatus, pumps for diffusion of medical substances, cochlear implants, and implanted biological sensors.

### BACKGROUND OF THE INVENTION

Active implantable medical devices typically comprise a generator that is to be connected electrically and mechanically to a probe, where the connection is made by the surgeon at the time of the implantation. More precisely, the generator includes a case containing the various electronic circuits and the power source of the device, and a connector head that is mechanically and electrically connected to the case and equipped with one or more cavities adapted to receive the probe(s).

The manufacture of the device generally comprises a series of stages, for example, initially, producing the complete case containing the electronic circuits and power source and having an upper face from which emerge a plurality of connector feed-through leads. The feedthrough leads are connected at one end to the electronic circuits inside the case and are intended to be connected to corresponding probe terminals in the connector head.

The connector head is then assembled on the case and mechanically joined to it, in particular by welding or soldering of the various terminals to the corresponding feed-through leads and/or by other techniques of mechanical connection to the respective elements, which techniques are common and known to persons of ordinary skill in the art, and include using an adhesive to join the connector head to the case.

The following stage is a coating stage in which the unit formed by the joined case and connector head is immersed in a silicone resin to ensure:

the joining of the connector head to the case, thus gaining an additional mechanical connection between these two elements,

a tight seal between the connector head and the case and around the various elements such as, in particular, the electric terminals, and

the electric insulation of the generator case on all or a portion of the external surface of the case, by coating of the unit.

The coating step is typically operated under a nitrogen atmosphere.

A mask, for example, a self-stick label, can be applied to a portion of the case that is not to be insulated when the body of the case is to be used as one of the electrodes for signal collection (detection) or for stimulation (delivering energy to the patient). The mask can then be removed after the immersion.

This assembly technique is a proven technique and an economical method. However, it presents in particular the disadvantage of a long time to manufacture, because it is necessary to await the complete drying of the adhesive before being able to pass to the following operations. More particularly, the stages of joining and coating generally each use a resin of the silicone type, for example, a resin of a type marketed by Rehau GmbH (Germany), which requires a reticulation for a typical length of time of 14 hours.

### OBJECTS AND SUMMARY OF THE INVENTION

The present invention proposes therefore to modify the existing process so as to reduce considerably, typically to a duration of a few minutes, the time required to carry out the stages of joining the connector head to the case and/or coating the case.

Primarily, the invention proposes to use, instead of the traditional silicone resin, a photo-reticulable (photo-hardening) resin, that can be reticulated with ultraviolet (“UV”) light, and to modify consequently the way in which the corresponding, joining and/or coating stages in the manufacturing process are carried out.

In one embodiment, the invention proposes a connector head comprising: means for the mechanical and electric assembly of the head onto a case of the device; a peripheral lip having a surface to join tightly to the case, so as to define an interface between the case and connector head including a closed volume able to receive a quantity of a photo-hardening adhesive; an opening for injection of the aforementioned adhesive, communicating with the closed volume; and at least one vent hole in communication with the closed volume.

The connector head is preferably made of a transparent or translucent material allowing the irradiation of the aforementioned quantity of adhesive by a light having a wavelength adapted to effect the hardening of the adhesive.

Advantageously, the opening for the injection of adhesive is located at one extremity of the closed volume and the vent hole(s) is/are located at the opposite extremity of the volume so that the adhesive can flow through and fill the closed volume prior to curing.

Another aspect of the invention is directed to a process of assembly characterized by the following stages: a) mechanical and electric assembly of the connector head onto the case forming an interface therebetween, the interface having a plane; b) injection of a quantity of photo-hardening adhesive, preferably a photo-reticulable silicone resin, in the interface between the case and the connector head; c) irradiation, essentially directed in the plane of the interface between the case and the connector head, of the quantity of adhesive by a light having a wavelength adapted to harden the adhesive; d) affixing a mask on a portion of the case; e) immersion of the case provided with its connector head in a photo-hardening resin, in particular a photo-reticulable silicone resin, so as to form on the case and the connector head a layer of photo-hardening resin coating; and f) irradiation of this coating layer of resin by a light having a wavelength adapted to harden the resin.

It should be understood that the masking step d) is optional, and when it is employed, the mask is removed after the immersion step e) and preferably prior to the irradiation step f).

### BRIEF DESCRIPTION OF THE DRAWINGS

Other features, characteristics and advantages of the present invention will appear to a person of ordinary skill in the art in view of the following detailed description made with reference to the drawings annexed, in which like reference characters refer to like elements, and in which:

FIG. 1 is a top, elevated perspective view of a case and connector head assembled in accordance with the present invention; and

FIG. 2 is a bottom perspective view of the connector head of FIG. 1.

### DETAILED DESCRIPTION OF THE DRAWINGS

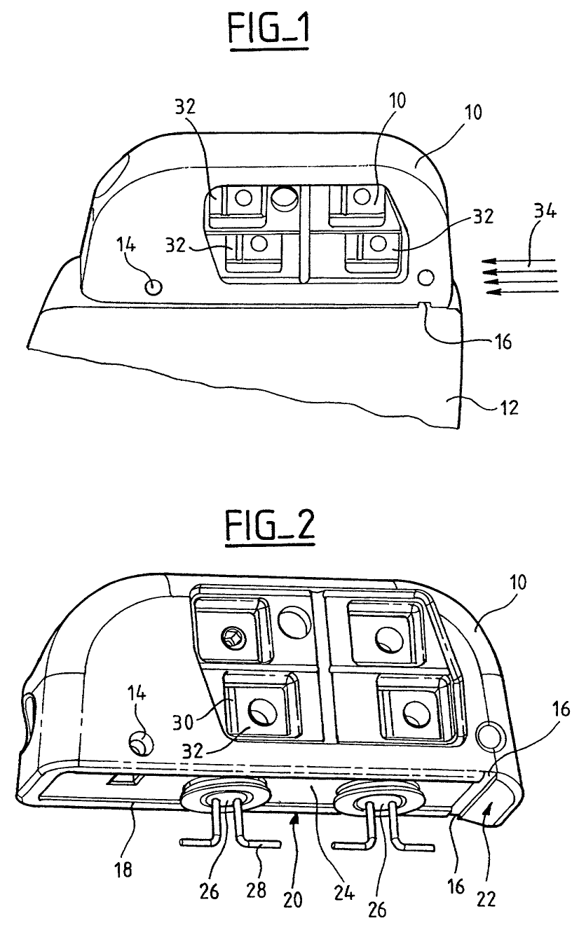

With reference to FIG. 1, a connector head 10 is illustrated joined to a case 12, such as a case of pacemaker. Head 10 is made of an insulating and translucent synthetic matter, which can be either a flexible or a rigid material. The material of this connector head is usually a silicone, in the case of a flexible head, or a polyurethane derivative, such as Tecothane, in the case of a rigid head. The case 12 of the generator is generally made out of titanium.

According to a preferred embodiment of the invention, a photo-reticulable silicone adhesive is used to join the connector head 10 onto the case 12, in particular an adhesive that can be reticulated with ultraviolet light (“UV”), such as adhesive No. 1138 marketed by Dymax, USA.

Once the connector head 10 is mechanically and electrically joined with case 12 (e.g., soldering feed-through lead 28 of insert 26 to a metallic contact for cavity 32), the adhesive is injected within the case-connector head interface, for example, by means of a syringe whose needle is introduced into an injection opening 14. Injection opening 14 is preferably located in the vicinity of the one of the extremities of the head 10 so that the operator can follow the progression of the front of adhesive along the interface (i.e., watch the adhesive flow through the interface), the head being a transparent or translucent material. The injection continues until the front of adhesive has arrived at the other extremity of the head, which can be detected by the exit of the adhesive through one or more vent holes 16 located at the extremity of the head opposed to the injection opening 14. Any surplus of adhesive coming out of opening(s) 16 can then be wiped off.

More precisely, with reference to FIG. 2, the closed volume receiving the adhesive is delimited by a peripheral edge 18 whose face 20, 22 is in contact with the case of generator, and mates with the “upper” surface of the case (not shown), so as to define a closed volume 24 communicating with outside only by injection opening 14 and vent hole(s) 16.

Volume 24 will thus be filled with adhesive, the adhesive circumventing and enveloping inserts 26 coming out from case 12, and thus ensuring the sealing around these elements. Inserts 26 carry electric feed-through leads or connectors 28 emerging into metallic contacts in areas 30 in the corresponding cavities 32 of the connector head 10. Cavities 32 include receptacles that will later receive the electric terminals of the probes (not shown). The unit is then closed by a final gluing of a closing cap or seal over the metallic contacts for cavity 32, thereby sealing the set screws used to secure the probes in the cavity 32 from bodily fluids (not represented).

Once the adhesive is injected into volume 24 in the manner indicated above, the connector head (a transparent or translucent material) is irradiated by a source of light having a wavelength that corresponds to that allowing the reticulation (curing/hardening) of the selected adhesive. Preferably, as indicated by arrows 34, the light beam is directed approximately according to the plane of the case connector head interface allow a better irradiation of volume 24 containing the adhesive; this irradiation is of course made possible by the transparent or translucent nature of material of the head.

The reticulation is thus obtained in a very short time, typically on the order of a few minutes, instead of the several hours (typically 14 hours) required with the traditional silicone adhesives used until now. The result is the connector head and case are formed as a unit.

The coating or immersion step, is carried out with a standard photo-reticulable silicone resin of the same type as the one that is used for the joining of the connector head.

The various steps of the stage of coating the unit are known and described above. Taking into account the various additional operations preceding and following the gluing and the coating steps in accordance with the present invention, the duration of a complete cycle of manufacture of a generator can be thus shortened by two to three days, with of course all the economic and technical advantages that result from this improvement.

One will note that the technique of the present invention can be transposed to manufacture of other elements of implantable devices, in particular for the gluing of the various elements of the stimulation probe.

One skilled in the art will appreciate that the present invention can be practiced by other than the described embodiments, which are presented for the purposes of illustration and not of limitation.

I claim:

1. A connector head for an active implantable medical device, such as a pacemaker, a defibrillator, a cardiovertor and/or a multisite device, comprising:
means for mechanically and electrically assembling said head onto a case of the device;
a peripheral lip having a surface to be mated to the case so as to define an interface between the case and the connector head comprising a closed volume able to receive a quantity of a photo-hardenable adhesive;
an injection opening communicating with said closed volume for receiving said adhesive; and
at least one vent hole communicating with said closed volume;
wherein said connector head comprises a material allowing the irradiation of said adhesive by a light having wavelength adapted to harden said adhesive.

2. The connector head of claim 1, wherein said closed volume has a first extremity and a second extremity, the second extremity being opposite to said first extremity, said injection opening is located at the first extremity and said at least one vent hole is located at the second extremity.

3. A process for assembling an active implantable medical device such as a pacemaker, a defibrillator, a cardiovertor and/or a multisite device comprising a connector head and a case, comprising the steps of:
a) providing a connector head with a first surface that defines an interface when connected to the case;
b) mechanically and electrically assembling the connector head onto the case to form a unit;
c) injecting a quantity of a photo-hardening adhesive into the interface between the case and the connector head;
d) irradiating said quantity of adhesive by a light having a wavelength adapted to harden the adhesive;
e) affixing a mask on a portion of the case;
f) immersing the case and connector head unit into a photo-hardening resin, so as to form thereon a layer of photo-hardening resin coating; and
g) irradiating said layer of resin with a light having a wavelength adapted to harden the resin.

4. The process of claim 3, wherein step b) further comprises injecting a photo-reticulable silicone resin.

5. The process of claim 3, wherein step c) further comprises primarily directing said light in the plane of the interface between the case and the connector head.

6. The process of claim 3, wherein step e) further comprises immersing said case and connector head unit in a photo-reticulable silicone resin.
